(19) United States
(12) Patent Application Publication
(10) Pub. No.: US 2025/0367119 A1
(43) Pub. Date: Dec. 4, 2025

(54) LUBRICANT

(71) Applicant: FM Vet Solutions, Dorset (GB)

(72) Inventors: Yoav ALONY-GILBOA, Dorset (GB); Anthony WILKINSON, Dorset (GB); Julian ALLEN, Dorset (GB); Helen ROGERS, Dorset (GB); Simon Edward UPTON, London (GB)

(21) Appl. No.: 19/224,698

(22) Filed: May 30, 2025

(30) Foreign Application Priority Data
May 31, 2024 (GB) 2407723.2

(51) Int. Cl. A61K 9/16; A61J 1/06; A61J 1/10; A61J 1/14; A61K 9/20

(52) U.S. Cl. CPC A61K 9/1641; A61J 1/067; A61J 1/10; A61J 1/1468; A61K 9/1623; A61K 9/1652; A61K 9/2018; A61K 9/2031; A61K 9/205

(57) ABSTRACT

The present invention relates to a dry powdered or tabletted lubricant precursor formulation comprising polyethylene glycol (PEG); sucrose and a thixotropic agent.

A physiologically acceptable water based lubricant comprising water and a dry powdered or tabletted lubricant precursor formulation, and a kit of parts for mixing and dispensing the lubricant.

### FIELD OF INVENTION

The present invention relates to a formulation for use in the preparation of a physiologically acceptable lubricant. The present invention also relates to a physiologically acceptable lubricant. The present invention also relates to a package for the delivery of the physiologically acceptable lubricant. The present invention also relates to a container for receiving the formulation and water to form the physiologically acceptable lubricant and to dispense this from the container.

### BACKGROUND OF INVENTION

Water based lubricants, such as a ready to use gel, are commonly used. This type of lubricant is often bulky, heavy and wasteful to transport and store.

This can be problematic, in particular when there is a requirement for a large volume of lubricant to be available because there may not be space to store the multiple containers required and it may be difficult to carry multiple containers to the place of use. Also ready to use gels are provided in single use containers therefore creating waste items that need to be disposed of.

The present invention provides a precursor formulation that can be mixed at the site of use in a reusable container.

The invention arose to overcome problems associated with prior delivery systems and products.

### PRIOR ART

UK Patent GB 2570955 (Alony-Gilboa, Quintana) discloses a package for delivery of a physiologically acceptable lubricant.

JorVet, 2017, J-lube jorvet.com/product/j-lube/(online) discloses a lubricant.

Vibenation Chemicals, 2023, Polyethylene Oxide (J-Lube) vibenationchemicals.com (online) discloses a lubricant.

Jorgensen Laboratories, 1995, J-lube Material Safety Data Sheet, vibenationchecminals.com/pages/msds (online) discloses a lubricant.

Chinese patent CN105695042B (SHANHAI JINZHAO ENERGY-SAVING TECH CO LTD) discloses a lubricant composition.

### SUMMARY OF INVENTION

According to a first aspect of the present invention, there is provided a dry powdered or tabletted lubricant precursor formulation comprising: polyethylene glycol (PEG); sucrose and a thixotropic agent.

Preferably, the formulation is powdered. Alternatively, the formulation may be an encapsulated powder or provided in a tabletted form.

The formulation is added to water to form a water-based physiologically acceptable lubricant.

The lubricant is suitable for animal or human use.

The inclusion of the thixotropic agent increases the viscosity of the lubricant so that the lubricant can be selectively dispensed from a container such as a bottle with a dispensing nozzle but does not flow freely from the nozzle. In this way the lubricant does not leak from the dispensing nozzle by simply inverting the bottle, instead the lubricant has to be urged from the bottle, for example by squeezing the bottle. The lubricant is also less liable to drip from a user's hands, from the patient and/or from equipment being used with the lubricant.

Advantageously this prevents unintended leaking, dropping or spilling of lubricant during use which can be a safety matter as spilt lubricant is a slip hazard. Furthermore, this prevents waste as leakage or spillage by the lubricant flowing too quickly from a container, or dripping from a user, patient or equipment is reduced or prevented so that less lubricant is used providing significant environmental benefits.

The composition of the formulation and the resulting viscosity of the lubricant is also such that it will form strings when dispensed, but the strings will break and not maintain a connection between the dispensing nozzle and the point of use. Advantageously this reduces the spread of lubricant to undesired places, for example from falling to the ground, on to a patient or onto the practitioner (user).

The thixotropic agent is preferably selected from one or more of: xanthan and/or agar.

In a preferred embodiment the formulation may include the addition of xanthan in an amount of between 1 wt % and 2 wt % and more preferably between 1.2 wt % and 1.6 wt %. Preferably the xanthan is an easy to disperse xanthan.

In a more preferred embodiment 1.6 wt % of xanthan is added to the formulation which is used to form a lubricant when added to water. The thixotropic properties of this lubricant with xanthan are particularly beneficial for use during examination of a patient enabling the examination to be completed with greater ease and using less lubricant, thus reducing waste and cost. This includes when performing surface examinations such as an ultrasound and also when performing internal examinations such as rectal and vaginal examinations typically as part of fertility examinations.

Without the presence of xanthan the lubricant has a viscosity that allows the lubricant to run easily (low viscosity) meaning that it will leak from a dispensing nozzle when it is inverted, even when the container/bottle is not squeezed. Furthermore when the lubricant of the present invention is being used it does not run off the hands or the examined surface, or any associated equipment in the same manner as known lubricants with lower viscosity that do not contain a thixotropic agent such as xanthan/agar. This again helps to reduce waste and spillage.

If the wt % of xanthan in the formulation exceeds 2 wt % the viscosity is too high so that the lubricant is congealed, it cannot be easily dispensed as a measured amount, and in use the lubricant does not spread easily and thus affects the ability to effectively perform the required procedure.

In another embodiment agar is included as the thixotropic agent in an amount of between 0.01 wt % and 5 wt % and preferably between 2.5 wt % and 5 wt %.

In a similar way without the presence of agar the lubricant viscosity is too low and if the wt % of agar in the formulation exceeds 5 wt %, the viscosity is too high for the controlled dispensing of a measured amount. In addition, when the viscosity is too high this affects spreadability of the lubricant, making it harder for the lubricant to be easily spread and thus affects the ability to effectively perform the procedure.

The molecular weight of the polyethylene glycol is preferably between 100,000 and 5,000,000.

In one embodiment, the polyethylene glycol is Polyox® WSR 301.

The formulation preferably comprises at least 5 wt %, preferably at least 10 wt %, for example about 11 wt % polyethylene glycol.

The formulation preferably comprises no more than 20 wt %, preferably no more than 15 wt % polyethylene glycol.

The formulation preferably comprises polyethylene glycol in an amount of between 10 wt % and 15 wt %, preferably 11.25 wt %.

The formulation preferably comprises no more than 95 wt % sucrose, preferably no more than 90 wt % sucrose, for example about 89 wt % sucrose.

The formulation preferably comprises at least 80 wt % sucrose, preferably at least 85 wt % sucrose.

The formulation preferably comprises sucrose in an amount of between 80 wt % and 95 wt %, preferably between 85 wt % and 90 wt %.

The sucrose is preferably powdered sucrose.

In one embodiment, the formulation is substantially free of silica.

The formulation preferably consists of: polyethylene glycol in the range of between 5 wt % and 15 wt %; sucrose in the range of between 80 wt % and 95 wt %; and xanthan between 1 wt % and 2 wt %.

In a preferred embodiment the formulation consists of a 11.25 wt % polyethylene glycol, 87.15 wt % sucrose and 1.6 wt % xanthan.

In another preferred embodiment, the formulation consists of 11.75 wt % polyethylene glycol and 86.65 wt % sucrose and 1.6 wt % xanthan.

According to a second aspect of the present invention, there is provided a physiologically acceptable water-based lubricant comprising water and a dry powdered or tabletted lubricant precursor formulation as herein described.

The lubricant may contain any suitable ratio of dry powdered or tabletted lubricant precursor formulation to water. Preferably, the ratio of dry powdered or tabletted lubricant precursor formulation to water within the lubricant is no more than 1:0.1; preferably no more than 1:0.05; for example, about 1:0.04. Preferably, the ratio of dry powdered or tabletted lubricant precursor formulation to water within the formulation is at least 1:0.01; preferably at least 1:0.02. Preferably the ratio of dry powdered or tabletted lubricant precursor formulation to water within the formulation is between 1:0.01 and 1:0.1; preferably between 1:0.02 and 1:0.05.

For example, the lubricant may be formed from 500 ml water with between 20 and 22 g of dry powdered or tabletted lubricant precursor formulation. The lubricant may be formed from 1000 ml water with between 20 and 22 g of dry powdered or tabletted lubricant precursor formulation.

According to a further aspect of the present invention, there is provided a package for delivery of a physiologically acceptable water based lubricant comprising: a dry powdered or tabletted lubricant precursor formulation as herein described housed in at least one capsule capable of disintegration on exposure to moisture, in which the or each capsule is encapsulated by an outer protective membrane configured in use to inhibit exposure of the capsule to moisture.

The capsule containing the dry powdered or tabletted lubricant precursor formulation is inserted to a container that contains or receives an aqueous solution that is typically water.

Preferably the container is a reusable bottle with a lid in which the formulation provided in a capsule and water can be mixed to form the lubricant wherein the capsule disintegrates and the formulation dissolves in the water to form the lubricant. The lubricant can then be dispensed from the reusable bottle for use. Advantageously this allows the lubricant to be made on site, which may be remote from the vehicle access point, for example on a farm, rather than carrying pre-mixed lubricant to site, saving space and unnecessary transport of water. Additionally, this allows for multiple doses of the formulation to be easily carried to the site of use and the lubricant only need be made up on site when required using a bottle and water provided on site.

This also reduces waste as the mixing and/or dispensing bottle can be reused, rather than single use containers that are typically used for ready to use lubricants.

Furthermore, when the dry formulation of the present invention is purchased, for example from wholesalers, the volume and weight of the purchased dry formulation is considerably less than that of a pre-mixed lubricant provided in a container ready for use. Therefore, storage, shipping and transport costs are reduced.

In a preferred embodiment the bottle is resiliently deformable at least in part and has a dispensing nozzle that enables lubricant to be dispensed when the bottle is squeezed. In this way a selected amount of lubricant can be quickly and easily dispensed by squeezing the bottle.

Preferably the bottle is transparent or translucent at least in part so that a user can view the level and consistency of contents.

Ideally the bottle has a volume of at least 500 ml to provide sufficient lubricant for standard procedures and a bottle size that is easy to carry and handle held in one hand for when dispensing.

In a preferred embodiment the bottle has a diameter of 68.30 mm and a height of 174.96 mm. The preferred wall thickness is 0.5 mm. The preferred opening diameter is 38 mm.

It is appreciated that the bottle may be provided in a range of sizes and dimensions.

The viscosity of the lubricant is such that lubricant will not be automatically released (pour freely) through the dispensing nozzle, even when the bottle is filled and inverted. The viscosity of the lubricant is such that the bottle must be squeezed to release the lubricant.

The dispensing nozzle is typically a narrowed or tapering opening to provide an exit for the lubricant from the container.

A problem with traditional lubricants is that the viscosity is low such that the lubricant flows freely from a dispensing unit so that too much lubricant may be expelled leading to waste, or so that lubricant may be spilt thereby causing a slip hazard.

The bottle preferably does not have a significantly long narrowed neck, and instead has an opening that widens to towards the bottle walls or has a neck similar to width of the body of the bottle so that a capsule can be dropped into the bottle through the opening and does not need to contact the neck or be forced through a long narrow neck. This also has the advantage of enabling the capsule to have minimal handling by a user and thus being less likely to commence disintegration due to moisture on a user's hands or the bottle neck and only disintegrate when in contact with water in the bottle.

In some preferred embodiments the dispensing nozzle includes at least one valve to assist with preventing erroneous dispensing of the lubricant and to assist with controlling the amount expelled. The valve may also act to enable a measured amount (dose) to be released on each squeeze of the bottle. The valve may also act to allow passage of air to enable the resiliently deformable bottle to reform after it has been squeezed to expel contents.

In another embodiment the nozzle is tapered and the opening dimensions of the nozzle is such that the lubricant will not drip from the nozzle unless force is applied due to the viscosity of the lubricant and its adherence to the wall of the nozzle.

In some embodiments the dispensing nozzle includes a lid or a cap so that the lubricant can be sealed in the container (bottle), for example for transport or to retain for use at a later time. The lid may be connected to the dispensing nozzle so that it remains associated even when the lid is displaced.

Preferably there is provided a kit of parts comprising a bottle with a dispensing nozzle and a capsule containing the formulation described above wherein the bottle receives at least one capsule and water in which the capsule disintegrates to release the formulation to the water to form a physiologically acceptable water-based lubricant.

In this way the lubricant is easily mixed on site in the bottle (container) and is selectively dispensed in doses through the dispensing nozzle by applying a squeezing force to the bottle.

In some embodiments the bottle may include incremental measure lines so that a user can monitor amount of lubricant dispensed and/or amount remaining. The measure line(s) may also be used to indicate the amount of water to be added to the container.

The capsule containing the dry powdered or tabletted lubricant precursor formulation may be inserted to a tract of a patient such as the birthing canal where the capsule disintegrates on contact with moisture in the birthing canal.

In a preferred embodiment the capsule is formed from materials that allows the capsule to disintegrate quickly (ideally within 30 seconds and preferably within 15 seconds) when inserted into the birth canal or added to water to make a liquid solution. It is appreciated that the capsule will disintegrate at different rates depending on temperature, disintegrating more quickly at warmer temperatures (typically over 25° C.) and more slowly at temperatures below this. Therefore, the lubricant can still be effectively formed even when only a cold water supply on site is available, or ambient temperatures are low, for example in winter.

The existing level of moisture when inserted into the birth canal will also affect the time it would take for the packet to disintegrate and powder to dissolve

The term “capsule” is used herein to refer to a container or envelope of suitable form for holding the dry powdered or tabletted lubricant precursor formulation.

The, or each, capsule is preferably in the form of an inner membrane. The inner membrane may be a sachet or bag.

The, or each, capsule is preferably formed at least in part of a bio-soluble polymer.

The bio-soluble polymer is preferably a heat sealable polymer.

The bio-soluble polymer preferably comprises polyvinyl alcohol.

The, or each, capsule preferably disintegrates within 15 seconds of exposure to water at a temperature of 25° C. or above. The capsule can still disintegrate at lower temperatures, but this will take more time.

In one embodiment, the outer protective membrane is preferably compostable.

The outer protective membrane preferably comprises at least one weakness line.

An outer protective membrane is arranged around the capsule to preserve the capsule and contents until use.

Preferably the protective membrane contains one capsule that contains one dose of formulation.

In some embodiments the outer protective membrane may be configured to receive and substantially encompass a plurality of capsules, for example to encapsulate two or more capsules, thereby having more than one standard dose in one protective membrane. This may be useful in situations where several “doses” of lubricant may be required or if wanting to pack multiple “capsules” in one compostable protective “outer” when transporting the product in large quantities, for example If to be provided as a “refill” capsule for creating lubrication on site in a multiple use bottle.

Preferred examples of the capsule are described in UK patent GB2570955B which discloses a package suitable for containing the lubricant described in this patent application. The capsule containing the formulation may either be placed in a container with water and mixed to form the lubricant prior to use, or the capsule containing the formulation may be inserted to the tract of a patient where contact with moisture in the tract will form the lubricant. It is appreciated that in some embodiments the capsule may have different properties more suited to either application.

According to a further aspect of the present invention, there is provided a method of delivering a physiologically acceptable water-based lubricant to an animal/human comprising: exposing a dry powdered or tabletted lubricant precursor formulation as herein described to a source of water, for example to an aqueous solution.

In the preferred embodiment, the aqueous solution is water.

The source of water may be provided at the site of administration of the precursor formulation, for example by moisture present on the adjacent surface of the body of the animal/human or water in a container that could be obtained on site from a mains water supply, or from onsite water storage such as a water butt.

The source of water may be provided as a solvent for preparing a lubricant from the precursor formulation.

The ratio of dry powdered or tabletted lubricant precursor formulation to water is at least 0.01 to 1, preferably at least 1:0.02. Preferably, the ratio of dry powdered or tabletted lubricant precursor formulation to water is no more than 1:0.1; preferably no more than 1:0.05; for example about 1:0.04. Preferably the ratio of dry powdered or tabletted lubricant precursor formulation to water is between 1:0.01 and 1:0.1; preferably between 1:0.02 and 1:0.05.

The method may further comprise: obtaining a package as herein described; removing the outer protective membrane of the package; and placing at least one of the capsules contained within the outer protective membrane at a predetermined location in relation to a source of water.

The at least one capsule may be placed in the predetermined location (such as in a birthing tract or in a bottle) and as the capsule disintegrates the dry powdered or tabletted lubricant precursor formulation becomes exposed to the source of water.

The shape and dimensions of the capsule is preferably selected to be suitable for ease of handling and placement, as well as being suitable to ideally contain one dose of the formulation.

In preferred embodiments the capsules are shaped and dimensioned to be received through an opening in a bottle, for example to be received through a neck of a bottle. For example, the capsule is ideally elongate having a length greater than its width to most easily be dropped through the neck of a bottle or any container. The capsule may preferably be rectangular or substantially tubular. The capsule(s) may be easily dispensed from the protective outer membrane containing the capsule through the neck of a bottle, that has or receives water, to form lubricant within the bottle, without contact with the hands of a user.

Preferably, the lubricant precursor formulation is in the range of between 10 g-100 g.

Preferably, a 20 g dose of lubricant precursor formulation may be provided within, for example, a 2.5 cm×5 cm capsule.

It is appreciated that a square capsule, for example 5 cm×5 cm may be provided and this can be folded and inserted through the neck of a bottle which preferably has a corresponding sized opening.

As mentioned above, lubricant may be formed by contact with fluid within a site usually in a body cavity of an animal/human wherein said fluid provides the source of water.

The body temperatures of most domestic animals, range between approximately 35° C. and 45° C. Most mammals have a body temperature no higher than 40° C. if healthy. It is known that the body temperature can vary with different species. Typically, the body temperature of a domestic animal is in the range of between 37° C. and 39° C. The site is for example a vaginal passage of an animal. The capsule is suitable for disintegrating within these temperature range, and typically the material of the capsule is intended to disintegrate most quickly within this temperature range to rapidly form a lubricant when used internally.

The lubricant precursor formulation, lubricant and/or packaging of the present invention may be used for any procedure which requires the lubrication of internal cavities or tracts.

The lubricant precursor formulation of the present invention when provided in a capsule may be easily located either in a container for mixing, or within a reproductive tract or rectum avoiding any spillage of the formulation.

Since the lubricant can also be mixed in a container with a water source a user can easily observe consistency of the lubricant prior to use and dispense the lubricant from the container as required.

The lubricant precursor formulation, lubricant and/or packaging of the present invention may typically be used in assisting birthing, for example, calving and lambing.

### BRIEF DESCRIPTION OF THE DRAWINGS

FIG. 1 schematically depicts a lubricant precursor housed in a capsule, wherein said capsule is encapsulated by an outer protective membrane;

FIG. 2 schematically depicts a plurality of capsules, each housing a lubricant precursor, encapsulated by an outer protective membrane; and

FIG. 3A shows an example of the container in the form of a bottle;

FIG. 3B shows a perspective view of the dispensing nozzle and lid with the lid displaced;

FIG. 3C shows a top view of the dispensing nozzle and lid with the lid displaced; and

FIG. 3D shows a cross section of the dispensing nozzle with a lid.

### DETAILED DESCRIPTION

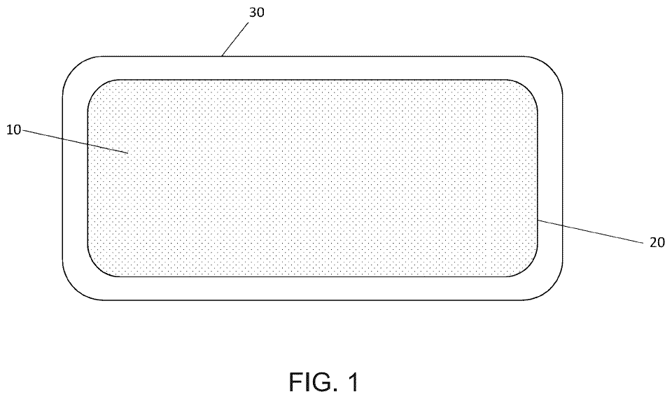

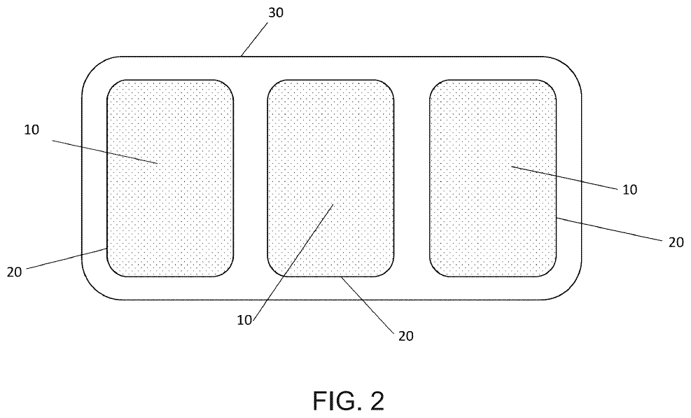

With reference to FIGS. 1 and 2, the package for delivery of a physiologically acceptable water based lubricant comprises: a dry powdered or tabletted lubricant precursor formulation 10 as herein described (referred to herein as “lubricant precursor”) housed in a capsule 20 which is capable of disintegration on exposure to moisture. The capsule 20 is encapsulated by an outer protective membrane 30 inhibiting exposure of the capsule 20 to moisture.

### Lubricant Precursor

While powder form lubricant precursors may be preferred in normal use (for example to provide for rapid dissolution in a source of water), tabletted precursors may provide some advantage in handling properties in some applications.

The lubricant precursor 10 comprises polyethylene glycol (for example Polyox® WSR 301), sucrose (preferably powdered sucrose) and thixotropic agent (preferably xanthan).

### Capsule

The capsule 20 is capable of disintegration on exposure to the physiological conditions provided at a predetermined site on a patient, or when exposed to a water source in a container, whilst also being physiologically acceptable.

The physiological conditions within the patient are typically “wet and warm”. The temperature of the predetermined site on the patient is preferably in the range of 35° C.-45° C.

A water source in a container may not be heated and may therefore be at an ambient temperature that is less than 35° C.

A physiologically acceptable material may be regarded as any material that does not cause any harm to the patient (for example does not product any toxic products) to which the material is being delivered. Materials that disintegrate on exposure to moisture at a temperature below 40° C. will be usable for most humans and domestic mammals. Materials that disintegrate on exposure to moisture at a temperature below 35° C. will be usable for all domestic animals and humans. However, materials that disintegrate when dry on exposure to temperatures below 35° C. may require refrigerated storage.

Solid or gel capsules may be used. The capsule 20 is preferably in membrane form. For example, the capsule 20 may be in the form of a bag or sachet.

The capsule 20 must disintegrate sufficiently to release the lubricant precursor when used, and this disintegration may be by at least partially dissolving, at least partially melting, or otherwise degrading once it has been inserted into the patient or exposed to water, ideally being submerged in water.

The capsule 20 may comprise materials that simply melt at temperatures equivalent to the temperature of the predetermined site on a patient (for example at temperatures of below 40° C.) and at the temperature of water available from a mains supply (for example tap or hose), or from water storage such as a water butt which typically ranges from around of 10° C. in winter (although lower temperatures may be experienced) and 20° C. in summer. To accommodate the temperature range expected from an onsite water supply, in some circumstances, the capsule 20 may require refrigeration during storage and transportation to ensure that the capsule 20 does not disintegrate prior to use.

Preferably therefore, the capsule 20 is of a material that dissolves (or melts) at least in part on exposure to moisture at a temperature in the range of 10° C.-45° C.

Bio-soluble polymers can provide such materials. Preferably, the capsule is formed from one or more bio-soluble polymers such as for example polyvinyl alcohol (PVOH or PVA). It is to be noted that other bio-soluble polymers, such as for example copolymers with PVOH/PVA may provide suitable materials for the capsule (either alone or in combination).

One or more bio-soluble polymers, such as for example PVOH/PVA, have been found to be suitable for forming thin membranes. Furthermore, one or more bio-soluble polymers have been found to have suitable heat sealing and adhesive properties. Physiologically acceptable adhesives are preferably used. One or more bio-soluble polymers are provided as thin membranes which are formed (by for example heat sealing and/or using physiologically acceptable adhesive(s)) into a sealed bag or sachet to provide the capsule 20 or inner membrane to encapsulate the lubricant precursor 10.

The thickness of the thin membrane and/or type of bio-soluble polymer (for example type of PVOH/PVA) is preferably chosen such that the capsule 20 does not start to dissolve if contacted with wet fingers of a user.

Preferably, a bio-soluble polymer(s) is selected which can fully dissolve at a wide range of temperatures.

Dissolution refers to the act of the capsule (for example membrane) going into the solution, such as for example into a patient's vaginal fluid or a bottle that contains water. Disintegration refers to the extent or rate at which the capsule (for example membrane) breaks up into smaller constituent parts.

It has been found that the relationship between capsule membrane thickness and dissolution time is linear.

It has however been found that the relationship between capsule membrane thickness and disintegration time is not necessarily linear.

It has been found that a 25 μm thick membrane comprising PVOH/PVA takes 90 seconds to dissolve and 2 seconds to disintegrate at 25° C. Furthermore, a 35 μm thick membrane comprising PVOH/PVA takes 100 seconds to dissolve and 3 seconds to disintegrate at 25° C.

Both dissolution and disintegration times will be faster when occurring at a patient's internal body temperature. For example, a healthy cow has a normal core body temperature around 38.6° C. so the dissolution time and disintegration time for the same 25 μm thick membrane will be shorter than at 25° C.

In order for the lubricant precursor 10 to escape the capsule 20, the capsule 20 must at least be partially disintegrated (by dissolution and/or disintegration).

There are four main factors to consider with regards to dissolution time: (i) the temperature of the liquid that the membrane is immersed in, (ii) the amount of fluid present if inserted into a body cavity. (iii) the type of material used to make the membrane and (iiii) the thickness of the membrane.

A controlled dissolution time of the capsule 20 may be advantageous as any medical procedure, for example obstetrical procedure, has to be done fairly rapidly and efficiently to reduce risk, for example to both dam and offspring, hence the need for a relatively quick lubricant precursor 10 release and adequate lubrication time (i.e. the time from the capsule 20 having been inserted to water or a site with moisture and the formation of the resulting lubricant that is formed to effectively reduce friction at the targeted location. A capsule 20 with too short a dissolution time may not allow the physician to sufficiently target the area that requires lubrication as it may cause the capsule 20 to dissolve prior to being correctly positioned.

The optimum size and shape of the capsule 20 may depend on the patient concerned and the dose of lubricant precursor to be delivered. The capsule is preferably rectangular or square in shape.

The capsule is preferably shaped and dimensioned to be received through an opening 102 in a bottle 100, for example to be received through a neck of a bottle. The capsule 20 is preferably elongate in shape to assist with ease of insertion through the neck of a bottle. For example, the membrane may be rectangular or substantially tubular.

The capsule may be easily dispensed from the outer protective membrane through the neck of a bottle, and into a water source, to form lubricant, without contact with the hands of a user.

During the predetermined procedure and depending on the type and/or size of patient, the person treating the patient (for example surgeon or farmer) may use a single capsule (for example sachet). The person treating the patient may however require a plurality of capsules in order to apply a suitable dose of lubricant to the predetermined site of the animal. For example, one capsule when added to the desired amount of water or inserted into the birth canal may be sufficient to provide a sufficient dose of lubricant to a ewe for parturition. In contrast, two or three capsules added to a corresponding amount of water or to the birth canal may be required in order to provide a sufficient dose of lubricant in a similar procedure for a cow.

### Outer Protective Membrane

As the capsule 20 is designed to disintegrate on exposure to moisture at a temperature below 45° C., it needs to be protected against dissolving, disintegrating and/or melting (depending on the material of the capsule 20 and the surrounding conditions) prior to application or insertion (for example during storage and/or transportation) into the patient.

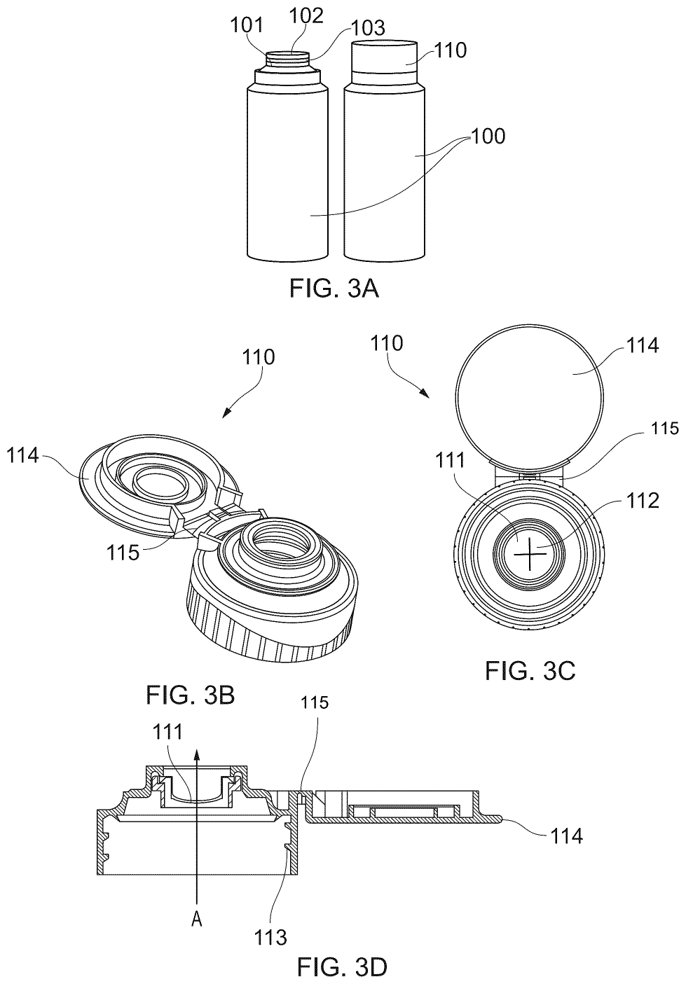

An outer protective membrane 30 (as shown in FIGS. 2-3) extends around and substantially encompasses the capsule 20. The outer protective membrane 30 is configured in use to protect the capsule 20 and to inhibit (preferably prevent) the capsule 20 from being contacted with moisture.

In use, the outer protective membrane 30 is removed or discarded prior to the application and/or insertion of the capsule 20 into the patient.

The outer protective membrane 30 may comprise a biodegradable and/or compostable material.

The outer protective membrane 30 is preferably configured from material which is suitable for blown film extrusion, to for example product bags.

In one embodiment, the outer protective membrane comprises Bio-Flex® FX 1130 produced by FKuR Kunststoff GmbH. Bio-Flex® FX 1130 is a biodegradable and compostable material which can be used for blown film extrusion to produce bags. Bio-Flex® is impermeable to moisture. An outer protective membrane 30 comprising Bio-Flex® will protect the capsule 20 and prevent the encapsulated capsule 20 from being contacted with moisture and as such prevent the capsule 20 from dissolving.

### Weakness/Ridged Edges

To improve efficient removal of the capsule 20 from the outer protective membrane 30 (and also to reduce any “wrap-rage”), the outer protective membrane 30 may comprise at least one weakness line for assisting removal of the outer protective membrane 30 to expose the at least one capsule 20. Optionally, or additionally, the at least one weakness line may comprise at least one perforation and/or tab which protrudes the outer protective membrane external surface for gripping and making it easier to remove the outer protective membrane 30. Optionally, or additionally, the outer protective membrane 30 may comprise at least one ridged edge. A ridged edge, similar to that of the ridged edges commonly used on confectionary wrappers, permits quick and simple removal of the outer protective membrane 30.

Each capsule 20 contains a dose of the lubricant precursor 10.

The capsule may be formed of a thin film having the same thickness (and therefore the same associated dissolution and/or disintegration time).

In one embodiment, the first and second capsule may be formed of a thin film having different thicknesses with respect to one another (resulting in different capsules having different dissolution times and/or disintegration times). For example, a first capsule encapsulated by the outer protective membrane 30 may be a first soluble capsule, and a second capsule encapsulated by the outer protective membrane 30 may be a second soluble capsule. The first soluble capsule may have a greater film thickness than the second soluble capsule. As a result, in use the first soluble capsule requires more time to dissolve and/or disintegrate than the second soluble capsule.

### Example 1—Preparation of a Dry Powdered or Tabletted Lubricant Precursor Formulation

A dry powdered or tabletted lubricant precursor formulation comprising polyethylene glycol (PEG); sucrose and a thixotropic agent when the formulation consists of 11.25 wt % polyethylene glycol, 87.15 wt % powdered sucrose and 1.6 wt % xanthan.

### Example 2—Preparation of a Dry Powdered or Tabletted Lubricant Precursor Formulation

A dry powdered or tabletted lubricant precursor formulation comprising 11.75 wt % polyethylene glycol, 86.65 wt % powdered sucrose and 1.6 wt % xanthan.

### Example 3—Preparation of Package

A capsule 20 was prepared from a film of PvOH/PVA having a thickness of 25 μm. The capsule 20 is a 5 cm×11 cm envelope defining a cavity. The capsule 20 is formed by sealing with adhesive (for example cyanoacrylate glue). The dry powdered or tabletted lubricant precursor formulation of Example 1 or Example 2 is received in the cavity and substantially encompassed by the capsule 20.

An outer protective membrane, in the form of a 6 cm×12 cm enveloped, was formed from a film of Bio-Flex® FX 1130 having a thickness of <110 mri. The envelope of the outer protective membrane defines a cavity within which the capsule is received and substantially encompassed.

A weakness line extending between adjacent or opposed edges of the outer protective membrane is provided to provide for ease of opening of the membrane and ease of removal of the capsule retained within the cavity.

In use, the user opens the outer protective membrane along the weakness line to access the capsule 20. The capsule 20 is contacted with a source of water either directly at the predetermined site or by dissolving the capsule in water to provide a lubricant for delivery to the predetermined site.

FIGS. 3A, 3B, 3C and 3C show an example of a bottle 100 and a corresponding dispensing nozzle 110 with a lid 114. These components are included in a kit of parts comprising a bottle 100 with a dispensing nozzle 110 and a capsule 20 containing the formulation 10.

The pictured bottle 100 holds 500 ml of liquid and has a resiliently deformable wall so that the bottle 100 can be squeezed to expel contents (not shown) from the dispending nozzle 110 through the valve 111.

The bottle 100 has neck 101 that tapers towards an opening 102. The opening 102 is closed by the dispending nozzle 110 and lid 114.

The dispensing nozzle 110 has a one way valve 111 that is a flexible layer with four displaceable flaps 112 that displace when the bottle is squeezed and liquid is squeezed through the valve 111 from the bottle 100. The valve 111 is arranged along a liquid pathway that is defined from the volume of the bottle 100, through the dispensing nozzle 110 indicated by the arrow ‘A’ on FIG. 3D.

The dispensing nozzle 110 is fitted to the neck 101 of the bottle 101 by a threaded connection 103 (bottle screw threads), 113 (dispending nozzle screw threads).

The dispensing nozzle 110 includes a lid 114 so that the valve 111 and thereby the fluid pathway A can be closed. This enables easy transportation of the lubricant and can enable the lubricant to be sealed and reused at a later time. The lid 114 is connected to the dispensing nozzle 110 by a hinge 115.

When preparing the lubricant the dispensing nozzle 110 is removed (FIG. 3A) and at least one capsule 20 (as shown in FIGS. 1 and 2) is inserted to the bottle 100. The bottle 100 may already be filled with water, or water may be added to the bottle 100 after the capsule 20 is inserted so that the capsule 20 disintegrates to release the formulation to the water to form a physiologically acceptable water-based lubricant.

The present invention provides a lubricant precursor which can be easily transported without contact with moisture. The present invention provides a lubricant precursor which can be applied efficiently and accurately once contacted with or exposed to a source of water to form a lubricant which can be applied to a desired location on the patient.

1. A dry powdered or tabletted lubricant precursor formulation comprising: polyethylene glycol (PEG); sucrose and a thixotropic agent.

2. A dry powdered or tabletted lubricant precursor formulation as claimed in a claim 1, in which the thixotropic agent is selected from one or more of: xanthan and agar.

3. A dry powdered or tabletted lubricant precursor formulation as claimed in claim 2 in which xanthan is included in an amount of between 1.2 wt % and 1.6 wt %.

4. A dry powdered or tabletted lubricant precursor formulation as claimed in claim 2 in which xanthan is included at 1.6 wt %.

5. A dry powdered or tabletted lubricant precursor formulation as claimed in claim 2 in which agar is included in an amount of between 0.01 wt % and 5 wt %.

6. A dry powdered or tabletted lubricant precursor formulation as claimed in claim 2 in which agar is included in an amount of between 2.5 wt % and 5 wt %.

7. A dry powdered or tabletted lubricant precursor formulation as claimed in a claim 1, in which the molecular weight of the polyethylene glycol is between 100,000 and 5,000,000.

8. A dry powdered or tabletted lubricant precursor formulation as claimed in claim 1, in which the formulation comprises at least 5 wt % polyethylene glycol.

9. A dry powdered or tabletted lubricant precursor formulation a claimed in claim 1, in which the formulation comprises no more than 20 wt % polyethylene glycol.

10. A dry powdered or tabletted lubricant precursor formulation as claimed in claim 1, in which the formulation comprises no more than 95 wt % sucrose.

11. A dry powdered or tabletted lubricant precursor formulation as claimed in a claim 1, in which the formulation comprises at least 80 wt % sucrose.

12. A dry powdered or tabletted lubricant precursor formulation as claimed claim 1, in which the formulation is free of silica.

13. A dry powdered or tabletted lubricant precursor formulation as claimed in a claim 1 in which the formulation consists of 11.25 wt % polyethylene glycol 87.15 wt % sucrose and 1.6 wt % xanthan.

14. A dry powdered or tabletted lubricant precursor formulation as claimed in claim 1 in which the formulation consists of 11.75 wt % polyethylene glycol 86.65 wt % sucrose and 1.6 wt % xanthan.

15. A physiologically acceptable water based lubricant comprising a mixture of water and a dry powdered or tabletted lubricant precursor formulation as claimed in a claim 1.

16. A physiologically acceptable water based lubricant as claimed in claim 15, in which the ratio of dry powdered or tabletted lubricant precursor formulation to water is no more than 1 part water: 0.1 part formulation.

17. A physiologically acceptable water based lubricant as claimed in claim 15, in which the ratio of dry powdered or tabletted lubricant precursor formulation to water is at least 1 part water: 0.01 part formulation.

18. A package for delivery of a physiologically acceptable water based lubricant comprising: a dry powdered or tabletted lubricant precursor formulation as claimed in claim 1 housed in at least one capsule capable of disintegration on exposure to moisture, in which the, or each, capsule is encapsulated by an outer protective membrane configured in use to inhibit exposure of the capsule to moisture.

19. A package as claimed in claim 18, wherein the, or each, capsule is in the form of an inner membrane.

20. A package as claimed in claim 19, wherein the inner membrane is a sachet or bag.

21. A package as claimed in claim 18, wherein the, or each, capsule is formed at least in part of a bio-soluble polymer.

22. A package as claimed in claim 21, wherein the bio-soluble polymer is a heat sealable polymer.

23. A package as claimed in claim 21, wherein the bio-soluble polymer comprises polyvinyl alcohol.

24. A package as claimed in claim 21, wherein the, or each, capsule disintegrates within 15 seconds of exposure to water at a temperature of 25° C. or above.

25. A package as claimed in claim 18, wherein the outer protective membrane is compostable.

26. A package as claimed in claim 18, wherein the outer protective membrane comprises at least one weakness line.

27. A package as claimed in claim 18, wherein the outer protective membrane comprises two or more capsules.

28. A kit of parts comprising a bottle with a dispensing nozzle and a capsule containing the formulation of claim 1; wherein, in use, the bottle receives at least one capsule and water in which the capsule disintegrates to release the formulation to the water to form a physiologically acceptable water-based lubricant.

29. A kit of parts according to claim 28 wherein the dispensing nozzle includes at least one valve.

30. A kit of parts according to claim 29 wherein the dispensing nozzle has a cap or lid.

31. A method of delivering a physiologically acceptable water-based lubricant to a patient comprising: exposing a dry powdered or tabletted lubricant precursor formulation as claimed in claim 1 to a source of water and administering the physiologically acceptable water-based lubricant to the patient.

32. A method as claimed in claim 31 wherein the ratio of dry powdered or tabletted lubricant precursor formulation to water is at least 0.01 parts water to 1 part precursor formulation.

33. A method as claimed in claim 31, in which the ratio of dry powdered or tabletted lubricant precursor formulation to water is no more than 0.1 parts water:1 part precursor formulation.

34. A method as claimed in a claim 31, further comprising: obtaining a package as claimed in claim 18; removing the outer protective membrane of the package; and placing at least one capsule into the water.

35. A method as claimed in claim 34, in which the at least one capsule is placed in the required position prior to exposing the dry powdered or tabletted lubricant precursor formulation to the water.
